(19) United States
(12) Patent Application Publication
(10) Pub. No.: US 2022/0244232 A1
(43) Pub. Date: Aug. 4, 2022

(54) METHOD FOR DETECTING AND QUANTIFYING LABILE ZINC

(71) Applicant: CITY UNIVERSITY OF HONG KONG, Kowloon (HK)

(72) Inventors: Wen-Xiong WANG, Kowloon (HK); Anqi SUN, Kowloon (HK)

(21) Appl. No.: 17/499,631

(22) Filed: Oct. 12, 2021

Related U.S. Application Data
(60) Provisional application No. 63/145,615, filed on Feb. 4, 2021.

(51) Int. Cl. G01N 33/18; G01N 21/64; C12N 1/16

(52) U.S. Cl. CPC G01N 33/1813; C12N 1/16; G01N 21/643

(57) ABSTRACT

Disclosed herein is directed to a method for detecting and quantifying labile zinc (Zn) ions in an aqueous sample. The method mainly includes steps of, constructing a standard curve of known concentrations of Zn ions versus fluorescence intensity of an adenine deficient (Ade(−)) yeast; preparing a mixture of the Ade(−) yeast, glucose and the aqueous sample and measuring the fluorescence intensity of the mixture; and determining the concentration of labile Zn ions in the aqueous sample by interpolation, in which the measured fluorescence intensity of the mixture is compared with that in the standard curve.

### CROSS-REFERENCES TO RELATED APPLICATIONS

This application claims priority and the benefit of U.S. Provisional Patent Application No. 63/145,615, filed Feb. 4, 2021, the entireties of which is incorporated herein by reference.

### BACKGROUND OF THE INVENTION

### Field of the Invention

The present disclosure relates to a method for detecting and quantifying labile zinc (Zn) ions in aqueous samples. More particularly, the disclosed invention relates to a method for detecting labile Zn ions in aqueous samples by use of adenine deficient (Ade(−)) yeasts.

### Description of Related Art

In general, to detect and quantify metals in the environment, conventional methods such as inductively coupled plasma/atomic emission spectrometry, atomic absorption spectrometry, microparticle-induced X-ray emission, synchrotron radiation X-ray spectrometry, cold vapor atomic fluorescence spectrometry, and electron paramagnetic resonance are generally employed for their high specificity and accuracy. However, these methods suffer from the drawbacks of the high cost and complicated procedures.

Excess of zinc (Zn) ions from natural or anthropogenic activities post threats to biota and to human health, especially in the case of labile Zn ions (Zn²⁺), which tend to bind with biomolecules. Therefore, quantifying labile Zn²⁺ in aqueous environments is important as their bioavailability are high. The design of organic fluorophores for Zn²⁺ detection is based on the reaction of fluorescein, quinolone, coumarins and naphthalene with Zn²⁺, and these fluorophores can potentially be used because of their high sensitivity and efficiency. However, limitations of organic fluorophores are obvious; for instances, the highest sensitivity of fluorescence probes is only effective within a narrow range of pH values, and specific chemosensors designed for Zn²⁺ are limited, resulting from the lack of intrinsic spectroscopic signals. Moreover, the specificity of chemosensors is interfered by other elements possessing similar chemical properties (e.g., Cd²⁺, Cu²⁺, and etc). As for other biosensors displaying high potential in quantifying Zn²⁺, they do exhibit several advantages, for example, both protein-based biosensors (e.g., enzymes, metalloproteins, and antibodies) and individual-based biosensors (e.g., engineered microorganisms) reveal high specificity, fast response, low cost, high portability, and ability to obtain real time signals in Zn²⁺ quantification, however, these biosensors suffer from the limitation in quantifying the trace amount of labile Zn²⁺.

In view of the foregoing, there exists in the related art a need of a novel method for effectively detecting and quantifying labile Zn²⁺ in the environment.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the present disclosure is directed to a method of detecting and quantifying labile zinc (Zn) ions in an aqueous sample. The method comprises: (a) constructing a standard curve of known concentrations of Zn ions versus fluorescence intensity of an adenine deficient (Ade(−)) yeast; (b) mixing the Ade(−) yeast, glucose and the aqueous sample and cultivating the mixture for at least 10 minutes; (c) measuring the fluorescence intensity of the mixture of the step (b); and (d) determining the concentration of labile Zn ions in the aqueous sample by interpolation, in which the measured fluorescence intensity of the step (c) is compared with that in the standard curve of the step (b).

According to some embodiments of the present disclosure, the Ade(−) yeast is produced by cultivating wild type Saccharomyces cerevisiae yeast in a medium comprising bacterial peptones, glucose, and yeast extracts for at least 24 hours. In one preferred embodiment, the bacterial peptones and the glucose are respectively present in the medium at the concentration of 20 g/L.

Optionally, the method of the present disclosure further comprises cultivating the Ade(−) yeast in a solution which contains 2.5 gram of glucose per liter prior to the commencement of the step (a).

According to some embodiments of the present disclosure, the aqueous sample has a pH value between 5 to 9. According to some other embodiments of the present disclosure, the aqueous sample has a salinity between 0.01-35 g/Kg. In still other embodiments of the present disclosure, the aqueous sample has one or more metal ions independently selected from the group consisting of Ag, Al, As, Ca, Cd, Co, Cu, Cr, Fe, Mg, Mn, Ni, Pb, Se, and Ti.

According to some embodiment of the present disclosure, the method of the present disclosure is capable of detecting labile Zn ions ranging from 0 to 0.5 μM. In preferred embodiments, the method of the present disclosure is capable of detecting labile Zn ions ranging from 0.01 to 0.1 μM.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a photograph depicting the result of observation of increased autofluorescence in yeast, the scale bar: 5 μm;

FIG. 2 is a line graph depicting linear relationship between concentrations of Zinc ions and fluorescence increase in YPD broth under different filter channels;

FIGS. 3A-3F respectively depicts the results of optimization tests for four factors, which are different growth phases (as depicted in FIGS. 3A and 3B), ratio of broth and water (as depicted in FIG. 3C), concentrations of glucose (as depicted in FIG. 3D), and different biomass (as depicted in FIGS. 3E and 3F);

FIG. 4 depicts the result of verifying whether the fluorescence increase is time dependent by Zn²⁺ addition (10 μM Zn²⁺, Excitation 488 nm);

FIG. 5 depicts the result of examining the relationship between fluorescence increase and labile Zinc ions at the concentration from 0 to 0.5 μM, [Zn²⁺]: concentration of Zn ions;

FIG. 6 depicts the result of a linear relationship between fluorescence increase and labile Zinc ions at the concentration from 0 to 0.1 μM;

FIG. 7 depicts the result of verifying the relationship between [Zn²⁺] and Zn accumulation in cells, the OD=0.03, in 2.5 g/L glucose medium;

FIG. 8 depicts the result of detecting labile Zinc ions in saline water;

FIGS. 9A-9F respectively depicts the results of detecting labile Zinc ions in aqueous solution imitating wastewater by adding metal ions, and the fluorescence intensity of Ade(−) yeast was detected in five channels;

FIG. 10 is a graph depicting the result of detecting labile Zinc ions in aqueous solution imitating wastewater by adjusting pH values; and

FIG. 11 depicts the result of detecting labile Zinc ions in a leachate sample.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### DEFINITIONS

For convenience, certain terms employed in the specification, examples and appended claims are collected here. Unless defined otherwise, all technical and scientific terms used herein have the same meaning as commonly understood by one of the ordinary skilled in the art to which this invention belongs.

The singular forms “a”, “and”, and “the” are used herein to include plural referents unless the context clearly dictates otherwise.

The term “aqueous sample” as used herein refers to a sample taken out from an aqueous solution, which is the one that the solvent is liquid water. An aqueous sample can be collected and/or obtained from natural water (e.g., rivers, streams, lakes, reservoirs, springs, seas, oceans, glaciers, and groundwater); drinking water such as tap water or filtered water; service water including domestic water, agricultural water, industrial water and commercial water; and wastewater generated from human activities. The aqueous samples can contain one or more substances including but not limiting to minerals, trace elements, metal ions and/or heavy metal ions, metabolite, excretion, microplastics, micronekton, and microorganisms. The aqueous sample has a variety of measurable parameters including but are not limited to pH value, and salinity.

### DETAIL DESCRIPTION OF PREFERRED EMBODIMENTS

The present disclosure is based, at least in part, on the discovery of a linear relationship between concentrations of zinc (Zn) ions and fluorescence intensity of cultivated adenine deficient (Ade(−)) yeasts. Hence, the Ade(−) yeasts may be used as a biosensor for detecting and quantifying a concentration of labile zinc ions ([Zn²⁺]) in a tested aqueous sample, in which the sensitivity and specificity of labile Zn²⁺ detection are greatly improved.

One aspect of the present disclosure is directed to a method for detecting and quantifying labile zinc (Zn) ions in an aqueous sample. The method comprises:

(a) constructing a standard curve of known concentrations of Zn ions versus fluorescence intensity of an adenine deficient (Ade(−)) yeast;

(b) mixing the Ade(−) yeast, glucose and the aqueous sample and cultivating the mixture for at least 10 minutes;

(c) measuring the fluorescence intensity of the mixture of the step (b); and

(d) determining the concentration of labile Zn ions in the aqueous sample by interpolation, in which the measured fluorescence intensity of the step (c) is compared with that in the standard curve of the step (a).

The method is composed by two parts, that is, standard curve construction steps and quantification steps. The standard curve is constructed based on the relationship between Zn ions' concentration and the corresponding florescence intensity of Ade(−) yeast (step (a)). In the step (a), the Ade(−) yeast are first produced and co-cultivated with various known concentrations of Zn²⁺ for a predetermined time, and the florescence intensities of the Ade(−) yeast under these known Zn²⁺ concentrations are measured, respectively. The Ade(−) yeast can be produced by any methods known to those skilled persons in the art, typically, Ade(−) yeast is produced by cultivating wild type yeast (Saccharomyces cerevisiae) strain in a yeast extract-based rich medium containing low level of adenine for certain period of time. According to the present disclosure, the Ade(−) yeast is produced by cultivating wild type yeast (i.e., strain W303) in a medium comprising bacterial peptones, glucose, and yeast extracts for at least 24 hours until the yeasts reach a stationary growth phase. In some embodiments, the cultivation lasts for at least 28 hours. According to some embodiments of the present disclosure, bacterial peptones and the glucose are independently present in the medium at a concentration from 0 to 50 g/L, for example, at 0, 2.5, 5, 10, 15, 20, 25, 30, 35, 40, 45, or 50g/L. In one working example, the bacterial peptones and the glucose are independently present in the medium at the concentration of 20 g/L.

According to some embodiments of the present disclosure, the concentration of Zn ions used for constructing the standard curve ranges from 0 to 20 μM; for example, about 0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, and/or 20 μM. In one working example, the standard curve is constructed with Zn²⁺ ([Zn²⁺]) ions at the concentrations of 0, 7.5, 12.5, 15, 17.5, and 20 μM. In another working example, the standard curve is constructed with Zn²⁺ ([Zn²⁺]) ions at the concentrations of 0, 0.01, 0.04, 0.06, 0.1, 0.2, 0.5, 0.6, 0.8, and 1 μM.

The fluorescence intensities of the Ade(−) yeast may be measured and determined by any means known in the art, specifically a flow cytometry. According to preferred embodiments, the Ade(−) yeast may be excited at an excitation wavelength between 350 nm to 500 nm, and fluorescence is measured at the emission wavelength between 450 nm to 800 nm. Each fluorescence intensities of the Ade(−) yeast corresponding to specific concentrations of Zn²⁺ are recorded and graphed to produce the standard curve. According to some embodiments, the standard curve is a linear standard curve with a correlation coefficient (R²) above 0.89, preferably, above 0.97.

In optional embodiments, before the commencement of the step (a), the method further comprises cultivating the Ade(−) yeast in a solution containing glucose at a concentration of 2.5 g/L. Specifically, the Ade(−) yeast may be cultivated in the glucose-contained solution for a period of time to allow the yeast cells to adapt to the glucose environment prior to the standard curve construction steps, thereby increasing reliability of measuring the fluorescence intensities of yeast cells.

The fluorescence intensity of Ade(−) yeast in an unknown aqueous sample may then be used to determine labile Zn ions therein with the aid of the standard curved constructed above. In the step (b), the Ade(−) yeast, glucose and the aqueous sample are mixed and cultivated for at least 10 minutes. According to some embodiments of the present disclosure, the aqueous sample has a pH value between 5 to 9; for example, a pH value of 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, or 9. In one working example, the aqueous sample has a pH value of 5.2 or 8.78. According to other embodiments of the present disclosure, the aqueous sample has a salinity of 0.01 g/Kg to 35 g/Kg; for example, a salinity of 0.01, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 g/Kg. In another working example, the aqueous sample has a salinity of 0.01, 1, 5, 10, 15, 20, 25, 25, or 35 g/Kg. The aqueous sample according to embodiments of the present disclosure may also comprise minerals, trace elements, metal ions and/or heavy metal ions therein. In one specific embodiment, the aqueous sample has one or more metal ions independently selected from the group consisting of Ag, Al, As, Ca, Cd, Co, Cu, Cr, Fe, Mg, Mn, Ni, Pb, Se, Ti and Zn. Examples of the aqueous sample suitable for use in the present method include, but are not limited to, a river water sample, a spring water sample, a stream water sample, a mountain water sample, a lake water sample, a groundwater sample, a rainwater sample, a seawater sample, a service water sample, and a wastewater sample. In one working example, the aqueous sample is collected from mountain water; in another working example, the aqueous sample is a seawater sample; and in further working example, the aqueous sample is a wastewater sample.

Then, in the step (c), the fluorescence intensity of the mixture of the step (b) is measured. As described in the step (a), the fluorescence intensity is measured and determined by any means known in the art, such as flow cytometer.

In the final step of quantification, i.e., the step (d), the concentration of labile Zn ions in the aqueous sample can be determined by interpolation from the standard curve constructed in the step (a). Specifically, the measured fluorescence intensity in the step (c) is substituted into the linear regression equation derived from the standard curve, thereby obtaining the concentration of labile

Taken together, the present method comprises at least, the steps (a) to (d) as described above, in which the present method is capable of detecting labile Zn²⁺ in any aqueous sample, particularly the zinc ions are present as a trace quantity. According to the present disclosure, the present method is capable of detecting labile Zn²⁺ ranging from 0 to 0.5 μM; for example, ranging from 0 to 0.45 μM, from 0 to 0.4 μM, from 0 to 0.35 μM, from 0 to 0.3 μM, from 0 to 0.25 μM, from 0 to 0.2 μM, from 0 to 0.15 μM, from 0 to 0.1 μM, from 0.01 to 0.09 μM, from 0.01 to 0.08 μM from 0.01 to 0.07 μM, from 0.01 to 0.06 μM, or from 0.01 to 0.05 μM. In some preferred examples, the present method is capable of detecting labile Zn²⁺ ranging from 0.01 to 0.1 μM.

By the virtue of the above features, the present method can detect and quantify environmental zinc ions, particularly the labile Zn ions in aqueous environments, which was unable to be detected by conventional detecting methodologies. In addition, the present method is capable of detecting labile Zn ions in aqueous samples that also contain a variety of substances, therefore can be applied in diverse water sources.

### EXAMPLES

### Materials and Methods

### Yeast cultivation and Determination of Fluorescence Intensity by Flow Cytometry

Wild type Saccharomyces cerevisiae (yeast; strain: W303) was used in this study. Cells were inoculated in yeast extract peptone dextrose (YPD) broth containing bacteriological peptones at 20 g/L, glucose at 20 g/L, and yeast extracts at 10 g/L (Sigma) at around 1.85×10⁵ cells/mL, and cultured (30° C., 200 rpm) for 24 h to obtain the adenine deficient (Ade((−)) yeast. Optical density (OD) values (600 nm) of yeast cells at different time points were measured by a microplate reader (FlexStation 3, Molecular Devices, USA) to develop the growth curve. Cells were obtained by centrifugation after 24 h (OD around 1.2) and washed by ultrapure water 3 times prior to the test.

For fluorescence observation, Ade(−) yeast cells were cultured in the medium with 10 μM Zn²⁺ for 10 min. A 0.5 mg/mL stock solution of concanavalin A (C2010, Sigma) was prepared and spread out on the dish to facilitate the immobilization of Ade(−) yeast on the culture dish. Fluorescence intensities of cells at channels including AF405, AF488 and AF633 were observed using a Celldiscoverer 7 Automated Microscope (Zeiss, USA). The location of mitochondria and nucleus was indicated by MitoTracker™ Deep Red FM (459 nM, Ex/Em-644/665 nm, M22426, Thermo Fisher Scientific, USA) and NucBlue™ Live ReadyProbes™ (2 drops per milliliter, Ex/Em-360/460 nm, R37605, Thermo Fisher Scientific, USA), respectively. For further determination of fluorescence intensity, Ade(−) yeast cells were cultivated alone or co-cultivated with various metals for 10 min, and the fluorescence intensity of 10,000 cells was recorded by flow cytometry (BD FACSAria™ III sorter, USA). Fluorescence intensity was recorded at filter channels including FSC, SSC, DAPI (Ex/Em 358/461 nm), Alex Fluor 430 (Ex/Em 434/540 nm), FITC (Ex/Em 494/519 nm), PE (Ex/Em 496/578 nm), PE-Texas Red (Ex/Em 496/615 nm), PerCP-Cy5-5 (Ex/Em 482/695 nm) and PE-Cy7 (Ex/Em 496/785 nm). The fluorescence increase (%) was calculated as the (fluorescence intensity in the test group-fluorescence intensity in the control group)/fluorescence intensity in the control group×100%.

### Quantification of Zn²⁺ in the Medium

The biomass of the Ade(−) yeast was diluted until OD value of 0.03 and were placed in glucose-based medium (2.5 g/L) and pre-cultured for 1 h. The medium was then replaced by a Zn²⁺ containing medium with the final concentrations of Zn²⁺ at 0, 0.01, 0.04, 0.06, 0.1, 0.2, 0.5, 0.6, 0.8, or 1 μM, followed by the detection of fluorescence intensity by flow cytometry after 1 h. Zn²⁺ at 0.1 μM was added to the medium to determine the reproducibility of different batches of yeast. To quantify the total Zn contents, Ade(−) yeast cells were cultured as described above, washed with ultrapure water for 4 times, digested with 1 mL 69% nitric acid (trace metal grade) and analyzed using ICP-MS (NexION 300X, PerkinElme USA). The detection limit of the concentration of Zn²⁺ (c_{L}) was obtained according to the International Union of Pure and Applied Chemistry:

$${c}_{L} = \frac{k {s}_{b 1}}{S}$$

where sh₁ and S represent the standard deviation of the blank sample and the sensitivity at low concentration (slope value of the standard curve with concentrations of Zn ranged from 0 to 0.1 μM), respectively, with k=3.

### Leachate Water Preparation

Mountain spring water was collected from the campus of The Hong Kong University of Science and Technology, using precleaned low density polyethylene (LDPE) bottle. Mountain water sample was transferred to a cooler at 4° C. immediately without any filtration and preconcentration treatments for subsequent experiment.

### Statistical Analysis

Data were expressed as the mean±standard deviation and performed in triplicate.

Statistical significance was determined using one-way analysis of variance and compared using LSD's test in SPSS 22.0.

### Example 1. The Autofluorescence of Adenine Deficient (Ade(−)) Yeast Increased with the Addition of Zn²⁺ Ions

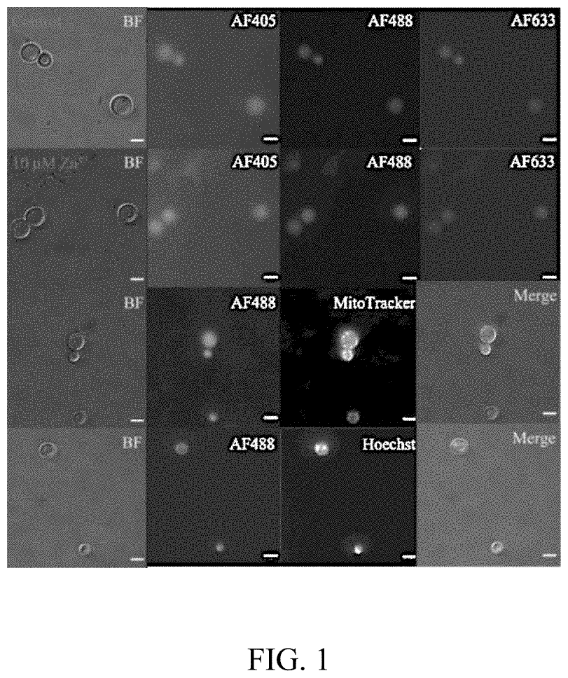

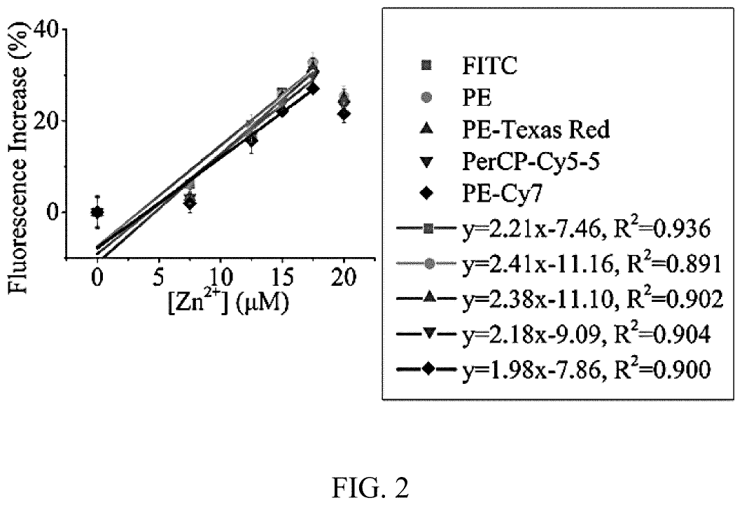

In this example, the intensity of autofluorescence of adenine deficient (Ade(−)) yeast was investigated with the addition of zinc ions, and results are provided in FIGS. 1 and 2.

To produce Ade(−) yeasts, the yeast strain W303 (wild type) was cultured in YPD broth for over 20 hours until a stationary phase with nearly unchanged OD values (around 1.3) was reached. Yeast W303 in this phase appeared to be red due to the accumulation of the red pigment (p-ribosylamino imidazole, AIR) in the adenine biosynthetic pathway (data not shown). Continuous consumption of nutrients from the medium led to the deficiency of adenine after cultivation for 20 h, further resulting in the necessity of synthesizing adenine intracellularly and the over accumulation of AIR. After reaching the stationary phase, it was found that a recession of red pigment was induced in Ade(−) yeast by adding Zn²⁺ (10 μM) within 10 minutes. The recession of red pigment was due to decreased synthesized AIR and the simultaneous transformation from AIR to adenine, suggesting that a side reaction was accelerated by Zn²⁺ and resulted in a reduction of AIR. An increased autofluorescence in the yeast W303 after addition of Zn²⁺ was observed under fluorescent microscopy, in which cells were excited by a 488 nm-laser (FIG. 1). Furthermore, by adding known concentrations of Zn ions (i.e., 0, 7.5, 12.5, 15, 17.5, and 20 μM) to the YPD medium, a linear relationship between [Zn²⁺] and fluorescence of Ade(−) yeast was observed (See FIG. 2). The results in FIGS. 1 and 2 suggested that the fluorescence intensity of Ade(−) yeast increased with the accumulation of zinc ions.

### Example 2. Construction of Standard Curve of Labile Zn²⁺ Concentration Versus Fluorescence Intensity of Ade(−) Yeast

As autofluorescence intensity of Ade(−) yeasts increased with the addition of Zn²⁺ ions (See Example 1), a standard curve of [Zn²⁺] and fluorescence intensity of Ade(−) yeasts may be established based on such relationship.

### 2.1 Optimization of Factors Influencing the Sensitivity of Ade(−) to Zn²⁺

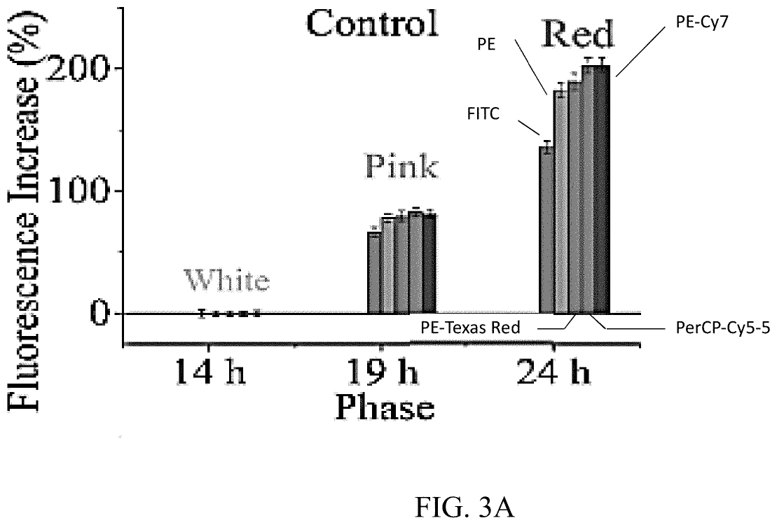

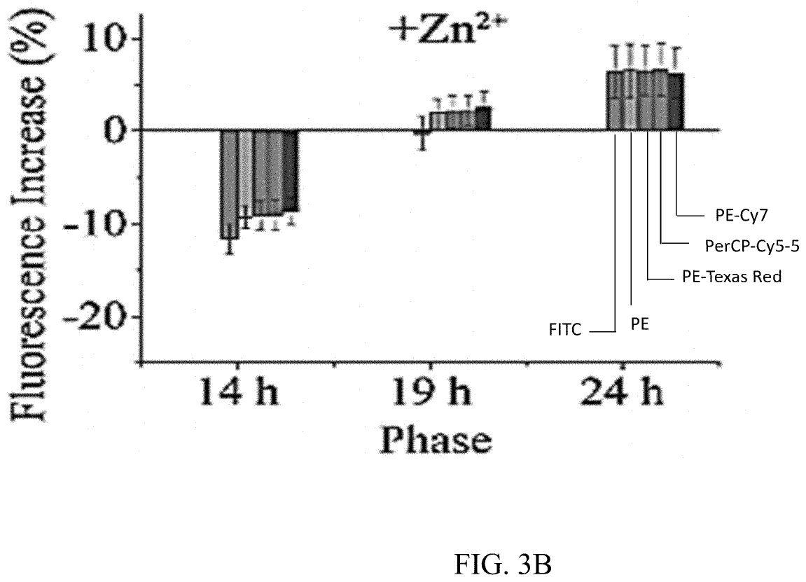

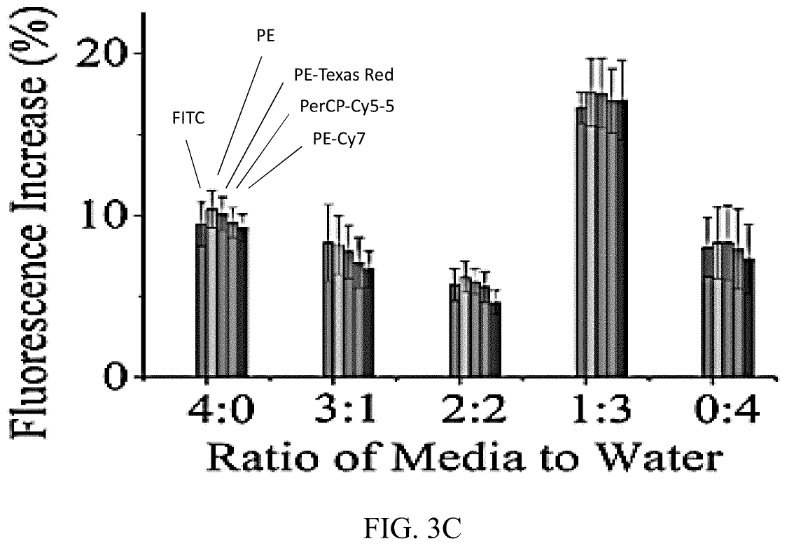

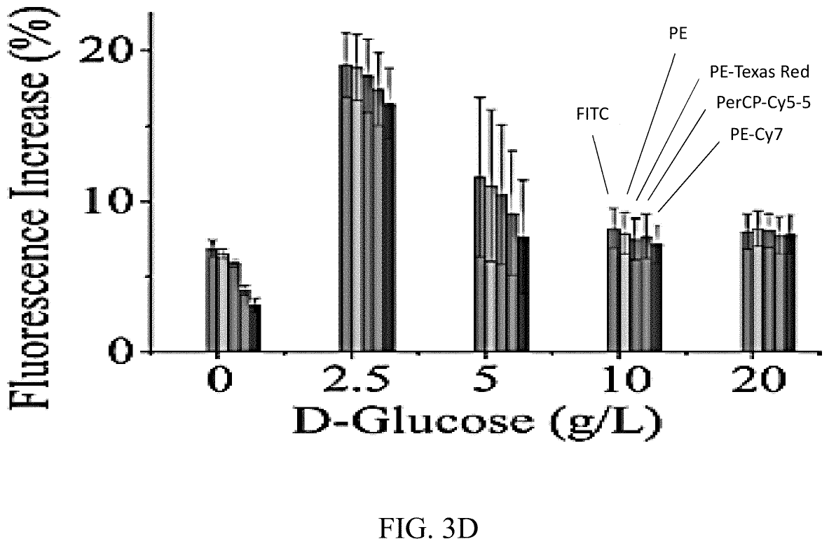

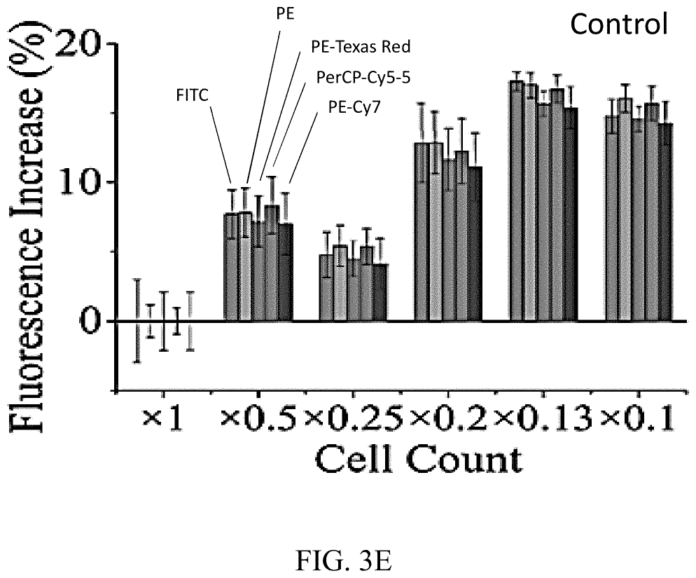

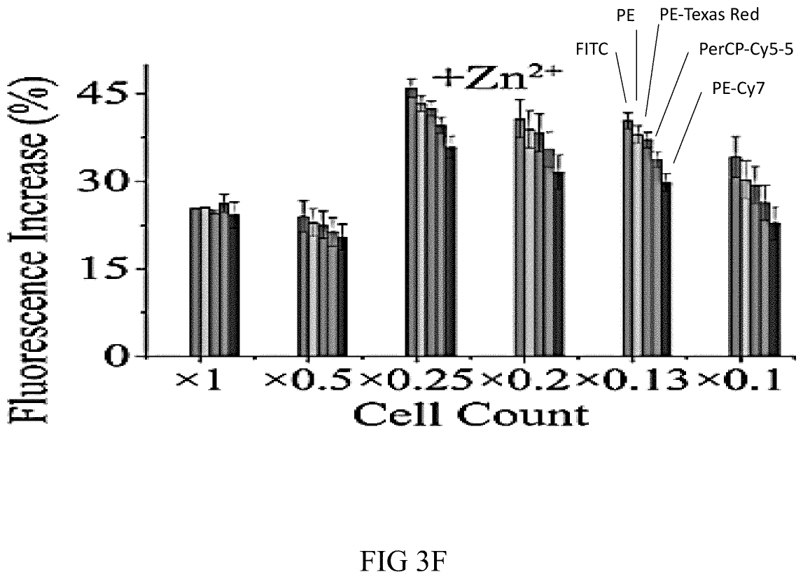

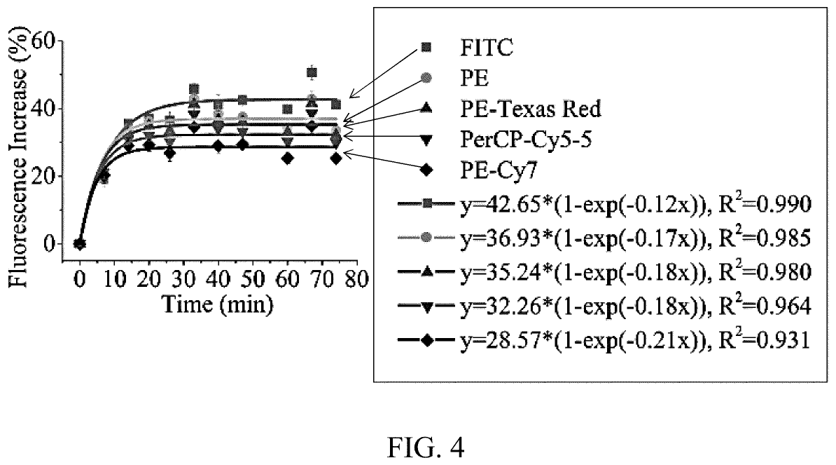

In this experiment, the effects of growth phase, biomass, media and time of Ade(−) on the sensitivity to Zn²⁺ ions were investigated. Yeast cells in different growth phases (e.g., cultivation for 14, 19 or 24 h) were collected and their fluorescence determined, respectively. A medium of mixed YPD broth and ultrapure water (i.e., 4:0/3:1/2:2/1:3/0:4) was used to culture Ade(−) yeast cells. Thus, the influence of ratio of YPD broth to water on Zn²⁺ directed fluorescence increase was determined. D-glucose was added in ultrapure water as the carbon source, with the final concentrations of glucose at 0, 2.5, 5, 10, or 20 g/L to determine the influence of glucose on fluorescence. Cells were diluted to obtain different biomass of cells at different OD values (i.e., around 1.2, 0.6, 0.3, 0.24, 0.16, 0.12). To verify whether the fluorescence increase is time dependent, flow cytometry was used to determine the fluorescence of cells after adding 10 μM Zn²⁺ in the medium, and the fluorescence intensity at different time points was recorded to explore the time dependent change of Zn²⁺ directed fluorescence increase. Results are depicted in FIGS. 3A-3D and 4.

Higher autofluorescence intensity was found in cells at the stationary phase after 24 h-culture with red pigment accumulation (FIGS. 3A and 3B). The gradual limited carbon source (i.e., decrease in the ratio of YPD broth to ultrapure water) did not change the Zn²⁺ directed fluorescence increases except for an obvious fluorescence increase when the ratio was 1:3 (FIG. 3C). As for the glucose test, the highest fluorescence increase directed by Zn²⁺ was observed when addition of glucose was set at 2.5 g/L (See FIG. 3D). As depicted in FIGS. 3E and 3F, the highest fluorescence increase was found when the biomass was diluted 4 times (10 μM Zn²⁺ for biomass with OD value around 0.3). Further, a time dependent increase of the fluorescence increase was found, and the fluorescence increase remained nearly unchanged after 10 min of exposure, suggesting that the shortest time for determining Zn²⁺ using this biosystem should be 10 min (FIG. 4).

### 2.2 Construction of Standard Curve of Fluorescence Intensity of Ade(−) Versus Zn²⁺

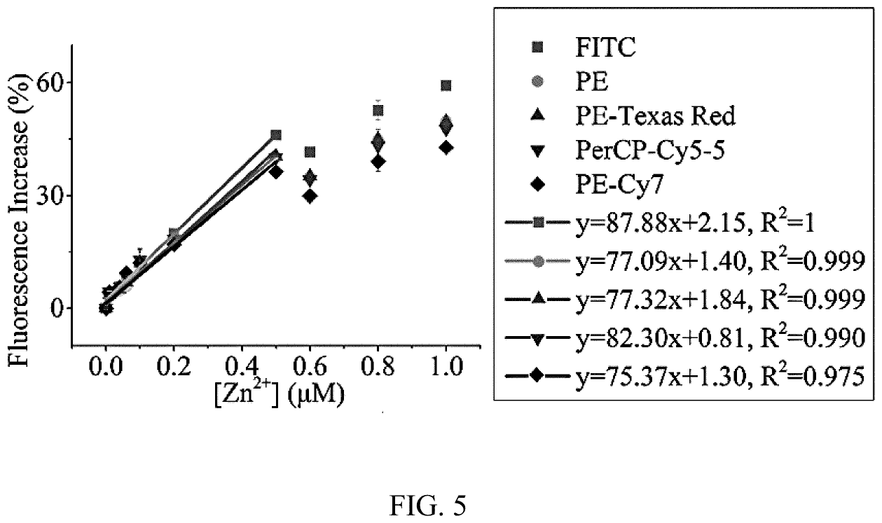

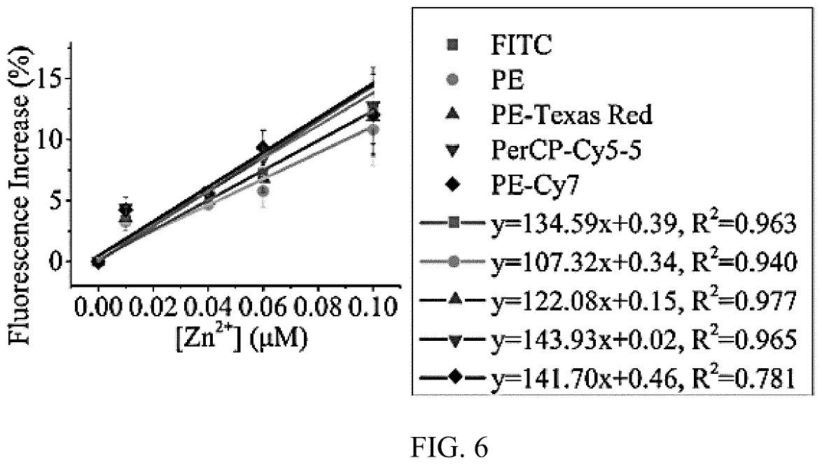

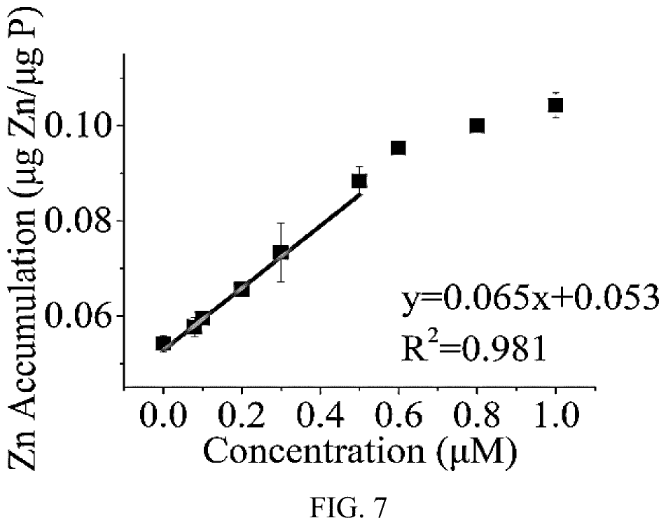

This experiment aimed to construct a standard curve of the [Zn²⁺] and the fluorescence of Ade(−) yeast. To this purpose, a Zn²⁺ containing medium with various final concentrations of Zn²⁺ (i.e., 0, 0.01, 0.04, 0.06, 0.1, 0.2, 0.5, 0.6, 0.8, or 1 μM) was used to culture the Ade(−) yeast cells (OD value=0.03), which was initially pre-cultured in a glucose-based medium (2.5 g/L) for 1 h. After another hour in culture, fluorescence intensity was detected by flow cytometry and a standard curve of [Zn²⁺] versus fluorescence of Ade(−) yeast was thus produced, as depicted in FIG. 5. Note that the fluorescence intensities under different filter channels varied, but the correlation between [Zn²⁺] and the fluorescence increase remained consistent (see, R²>0.975, as depicted in FIG. 5). Further, a positive correlation was found between fluorescence and [Zn²⁺] ranged between 0 to 0.5 μM (see, FIG. 5), even between 0 to 0.1 μM (see, FIG. 6), suggesting that it was possible to use the Ade(−) yeast cell as a sensitive biosensor to detect labile Zn²⁺ less than 0.5 μM. The strict correlation between bioaccumulated Zn²⁺ and extracellular [Zn²⁺] was found when [Zn²⁺] was 0-0.5 μM (R²=0.981, as depicted in FIG. 7), which was consistent with the above-mentioned correlation.

### Example 3. Detection and Quantification of Labile Zn²⁺ in Aqueous Samples

In this experiment, the specificity of [Zn²⁺] directed fluorescence intensity increase of Ade(−) yeasts in various aqueous samples (e.g., seawater, wastewater and natural leachate) was investigated. Ideally, if the specificity is high, then the Ade(−) yeasts may serve as a universal detector for different water environment.

### 3. 1 Seawater

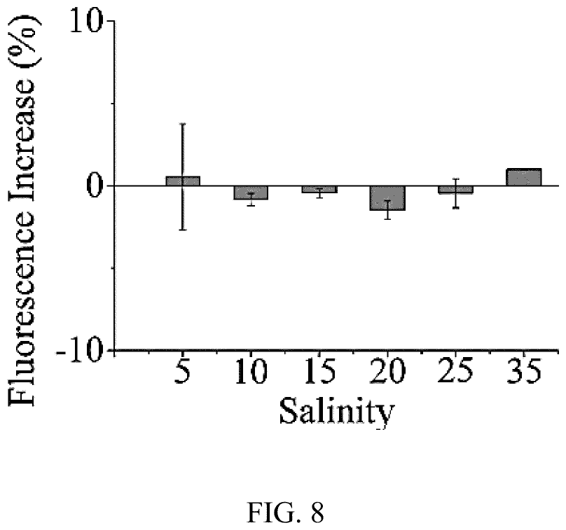

To mimic the saline water environments, NaCl was added to produce water solution with different salinities (i.e., 5, 10, 15, 20, 25 and 35 g/Kg). In this study, Ade(−) yeast cells were firstly cultured in 2.5 g/L glucose for 1 h, then were transferred to each medium with different salinity and 0.5 μM of Zn²⁺. The fluorescence intensity of Ade(−) yeast cells cultivated in medium only with 0.5 μM Zn²⁺ and no salinity was regarded as the control. After culturing in the adjusted medium (i.e., having various salinity) for 1 h, the fluorescence intensity of Ade(−) yeast cells was recorded by flow cytometry. Result is depicted in FIG. 8.

The data depicted in FIG. 8 showed that the Ade(−) yeast's tolerance to salinity was high, particularly when the salinity was similar to that of the seawater (around 35 g/Kg), suggesting that Ade(−) yeast has a potential to detect and quantify Zn²⁺ in seawater sample.

### 3.2 Wastewater

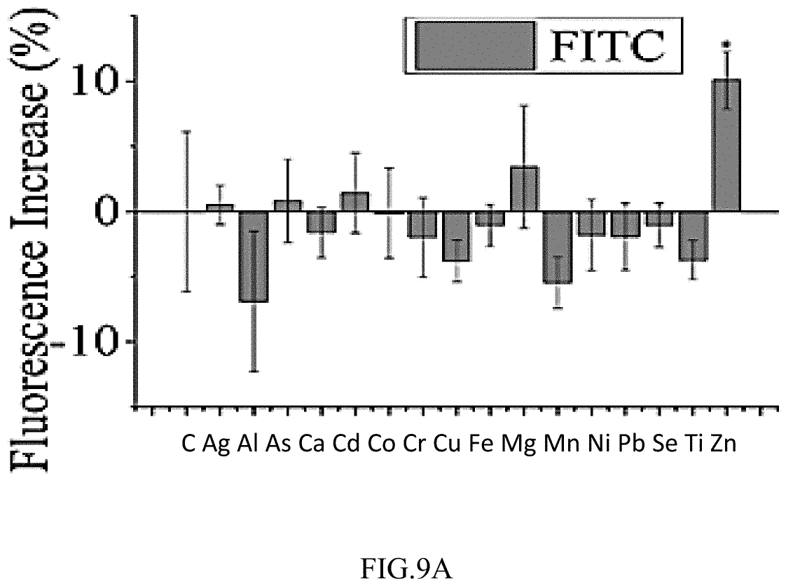

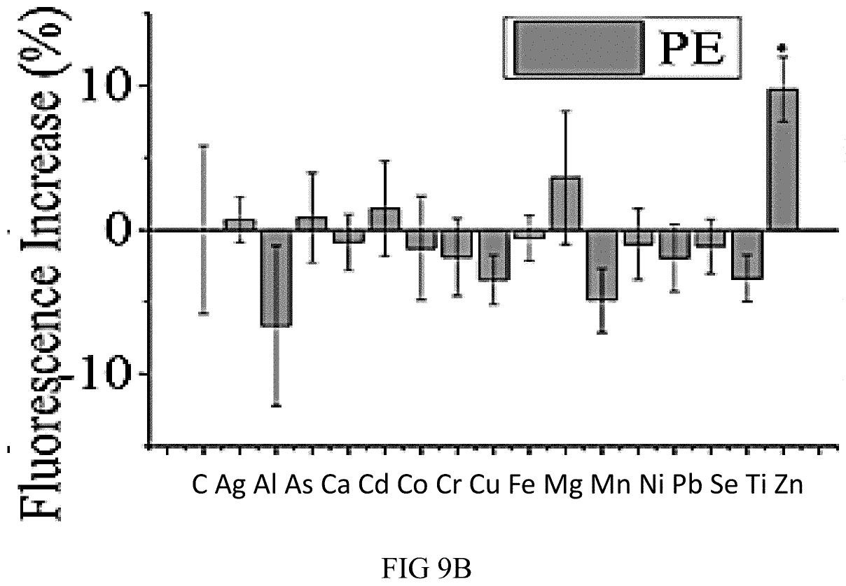

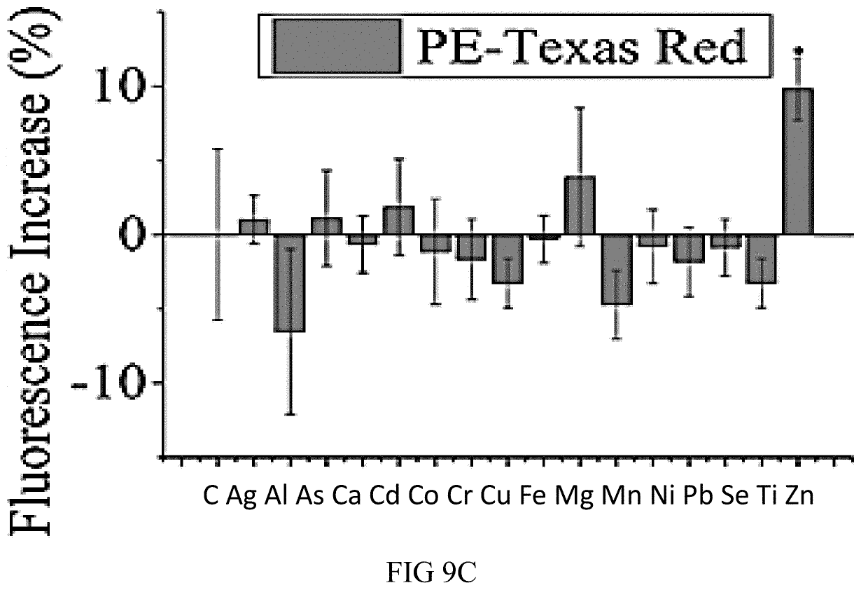

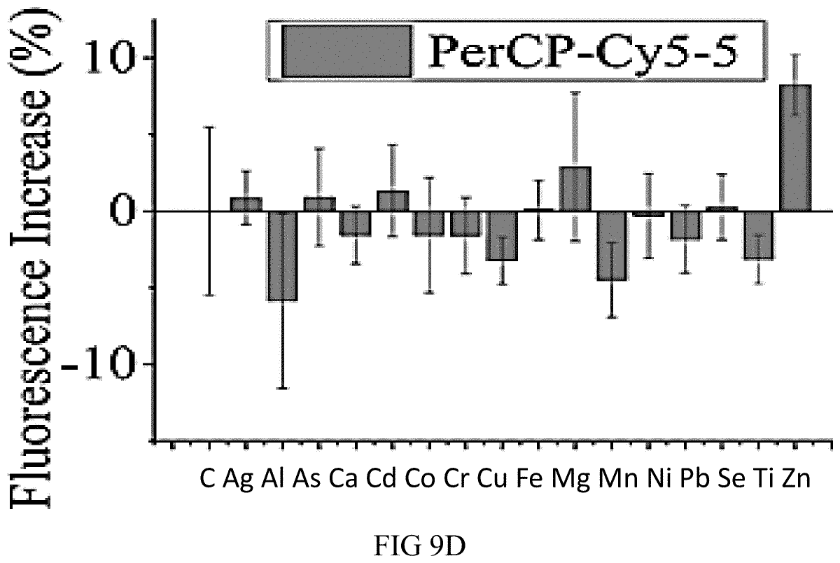

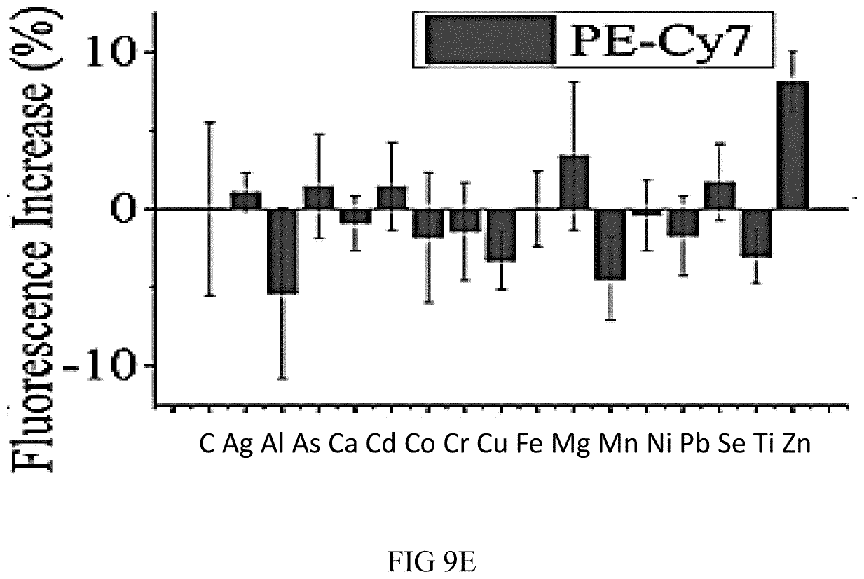

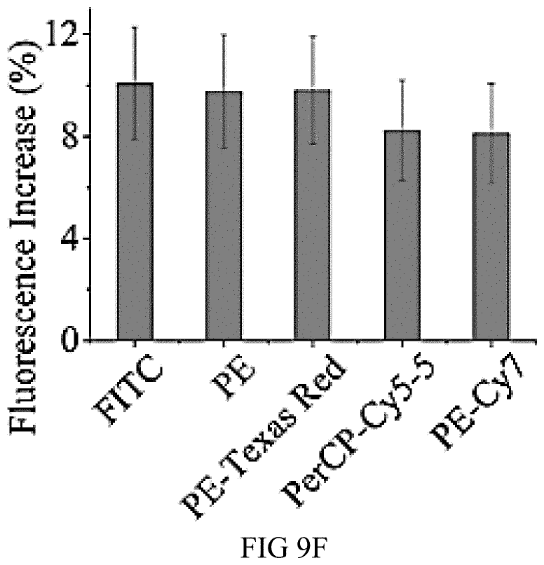

To imitate the contents of wastewater, Ade(−) yeast cells were cultured in YPD broth and various types of metals including Ag, Al, As, Ca, Cd, Co, Cr, Cu, Fe, Mg, Mn, Ni, Pb, Se, Ti, and Zn were independently added at 10 μM. After co-culturing for 10 min, the fluorescence intensity of 10,000 cells was recorded by flow cytometry, results were depicted in FIGS. 9A to 9F. It was found that the fluorescence intensity in 488 nm-laser excited channels increased significantly with the addition of Zn²⁺ ions, while other metal ions did not induce significant autofluorescence change in Ade(−) yeast regardless under which filter channel (see FIGS. 9A-9F). This result suggests a possible practical use of Ade(−) yeast in quantifying [Zn²⁺] specifically in wastewater, particularly those contaminated by heavy metal ions.

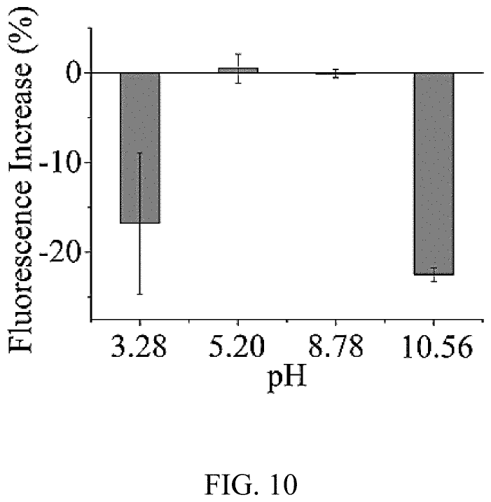

In addition, the effect of pH on the specificity of the Zn²⁺ directed fluorescence increase in Ade(−) yeasts was investigated. To this purpose, Ade(−) yeasts were pretreated in 2.5 g/L glucose solution for 1 h, then were transferred to four solutions independently containing 2.5 g/L glucose and the pH value adjusted to 3.28, 5.20, 8.78 or 10.56 with 1 M NaOH and 1 M HNO₃. After cultivating in the pH adjusted solution for another 1 h, the fluorescence intensity of Ade(−) yeast cells was recorded by flow cytometry. As depicted in FIG. 10, the sensitivity to Zn²⁺ remained consistent when the pH value changed from 5.20 to 8.78, suggesting the Ade(−) yeasts were capable of detecting Zn²⁺ ions in weak acidic to weak alkaline environments.

### 3.3 Leachate Water

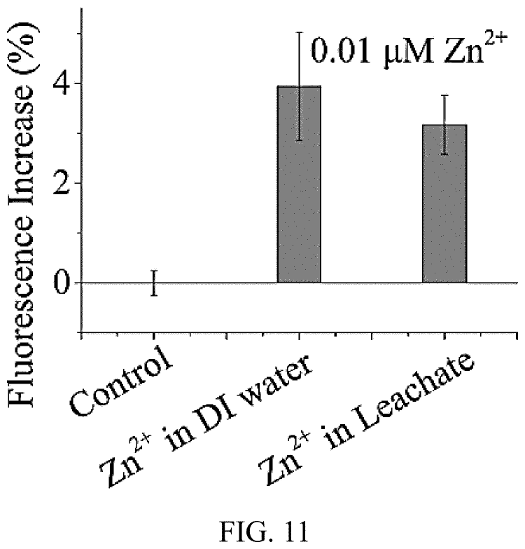

In this experiment, mountain water was collected to explore the possible practical use of Ade(−) yeasts in quantifying labile Zn²⁺. After culturing in 2.5 g/L glucose solution for 1 h-pretreatment, Ade(−) yeast cells were collected and transferred to the mountain water sample solution, in which 2.5 g/L glucose was added. Additionally, 0.01 μM Zn²⁺ was added as the internal standard to determine the possible influence of components in mountain water on the sensitivity of Ade(−) yeast cells to Zn²⁺. The culture time was limited to shorter than 15 min to avoid any unwanted interference induced by other metal ions in the natural mountain water. Following culturing for 1 h, flow cytometry was used to detect the fluorescence intensity of yeast cells. To quantify the total Zn content in the mountain water, 1 mL of the water was mixed with 1 mL of 10% HNO₃ and heated at 80° C. for 24 h, then was quantified by ICP-MS (NexION 300X, Perkin Elmer, USA). Results are depicted in FIG. 11.

The data in FIG. 11 showed that intrinsic components in the mountain water did not affect the detection accuracy of Zn²⁺ at 0.01 μM. Moreover, the organic substances in the mountain water would not affect the uptake of added Zn²⁺, indicating that the high ability of Ade(−) yeast cells to deprive labile Zn²⁺ from nonspecific adsorption on these organic matters, therefore rendering the Ade(−) yeasts as a stable indicator for detecting the labile Zn²⁺ in natural water source.

Taken together, the fluorescence intensity of Ade(−) yeast can be used to detect and quantify labile Zn²⁺ that are in trace amount (e.g., lower than 0.1 μM in the present disclosure) without being interfered by other elements and substances (e.g., metal ions and solutes in aqueous solution).

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for detecting and quantifying labile zinc (Zn) ions in an aqueous sample, comprising:
(a) constructing a standard curve of known concentrations of Zn ions versus fluorescence intensity of an adenine deficient (Ade(−)) yeast;
(b) mixing the Ade(−) yeast, glucose and the aqueous sample and cultivating the mixture for at least 10 minutes;
(c) measuring the fluorescence intensity of the mixture of the step (b); and
(d) determining the concentration of labile Zn ions in the aqueous sample by interpolation, in which the measured fluorescence intensity of the step (c) is compared with that in the standard curve of the step (a).

2. The method of claim 1, wherein the Ade(−) yeast is produced by cultivating wild type Saccharomyces cerevisiae yeast in a medium comprising bacterial peptones, glucose, and yeast extracts for at least 24 hours.

3. The method of claim 2, wherein the bacterial peptone and the glucose are respectively present in the medium at the concentration of 20 g/L.

4. The method of claim 3, further comprising cultivating the Ade(−) yeast in a solution containing 2.5 g glucose/L prior to the commencement of the step (a).

5. The method of claim 1, wherein the aqueous sample has a pH value between 5 to 9.

6. The method of claim 1, wherein the aqueous sample has a salinity between 0.01-35 g/Kg.

7. The method of claim 1, wherein the aqueous sample has one or more metal ions selected from the group consisting of Ag, Al, As, Ca, Cd, Co, Cu, Cr, Fe, Mg, Mn, Ni, Pb, Se and Ti.

8. The method of claim 1, wherein the method is capable of detecting labile Zn ions ranging from 0 to 0.5 μM.

9. The method of claim 8, wherein the method is capable of detecting labile Zn ions ranging from 0.01 to 0.1 μM.
